(19) United States
(12) Patent Application Publication
(10) Pub. No.: US 2007/0081972 A1
(43) Pub. Date: Apr. 12, 2007

(54) Polymer-based delivery system for immunotherapy of cancer

(75) Inventors: Anthony D. Sandler, Bethesda, MD (US); Aliasger K. Salem, Coralville, IA (US)

Correspondence Address: FULBRIGHT & JAWORSKI L.L.P., 600 CONGRESS AVE., SUITE 2400, AUSTIN, TX, 78701, US

(73) Assignee: The University of Iowa Research Foundation

(21) Appl. No.: 11/527,746

(22) Filed: Sep. 26, 2006

Related U.S. Application Data
(60) Provisional application No. 60/722,283, filed on Sep. 30, 2005.

(51) Int. Cl. A61K 48/00; A61K 38/19; A61K 38/20; A61K 38/21; A61K 39/02; A61K 39/00; A61K 9/50; A61K 9/16

(52) U.S. Cl. 424/85.1; 424/490; 514/44; 424/234.1; 424/277.1; 977/906; 424/85.2; 424/85.6; 424/85.7; 514/54

(57) ABSTRACT

The present invention relates to the treatment of cancer using a polymer-based delivery system to provide a plurality of tumor cell antigens to a immunocompetent subject in conjunction with an immunodulatory substance.

### BACKGROUND OF THE INVENTION

The present application claims benefit of priority to U.S. Provisional Application Ser. No. 60/722,283, filed Sep. 30, 2005, the entire contents of which are hereby incorporated by reference.

I. Field of the Invention

The present invention relates to the fields of oncology, immunology and biology. More particularly, the invention relates to the delivery of tumor cell lysates using polymers and immunomodulators.

II. Related Art

Cancer constitutes one of the greatest health threats in the world, responsible for over one-half million deaths each year in the U.S. alone. Unfortunately, current treatment methods for cancer, including radiation therapy, surgery, and chemotherapy, are known to have limited effectiveness. New and improved methods of cancer therapy are therefore desired. Immunotherapy is promising new form of cancer treatment.

Cancer immunotherapy involves recruitment of the host's immune system to fight cancer. The central concept relies on stimulating the patient's immune system to attack tumor cells. Normally, the immune system responds to invasion on the basis of discrimination between self and non-self, but many kinds of tumor cells are tolerated by the patient's immune system, at least in part due to the fact that cells are essentially the patient's own cells. However, many kinds of tumor cells display unusual antigens that are not normally present on that type of cell. These antigens make ideal candidate targets for the immune system.

Antibodies are one component of the adaptive immune response, recognizing foreign antigens and stimulating an immune response to them. A number of immunotherapeutic approaches to the treatment of cancer involve the use of antibodies. In particular, monoclonal antibodies make it possible to raise antibodies against specific tumor target antigens. Herceptin is an antibody against ErbB2 and was one of the first generation of immunotherapeutic treatments for breast cancer. However, the number of appropriate targets, and the corresponding development of safe and effective antibody therapeutics, has so far been limited.

Other types of immunotherapy also exist. For example, cytokines, such as IL-2, play a key role in modulating the immune response, and have used in conjunction with antibodies in order to generate a greater immune response. Unfortunately, the administration of such cytokines may cause systemic inflammation, resulting in serious side effects and toxicity. Yet another form of immunotherapy involves tumor vaccines. A large number of these vaccines, which involve the administration of either tumor antigens or genetics sequences encoding such antigens, have been attempted. However, tumor antigen variation and lack of immunogenicity still hamper this approach. Thus, new and improved immunotherapies for the treatment of cancer are desired.

### SUMMARY OF THE INVENTION

In accordance with the present invention, there is provided a method of treating or preventing cancer in a subject comprising administering to said subject a composition comprising a biocompatible polymer, a plurality of tumor cell antigens and an immunostimulatory agent. The biocompatible polymer may comprise silk, elastin, chitin, chitosan, poly(d-hydroxy acid), poly(anhydrides), and poly(athoesters). More particularly, the biocompatible polymer may comprises polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis(p-carboxypheonoxy) methane] (PCPM), copolymers of SA, CPP and CPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, polylactide-co-glycolide, polylactic acid, and polyethylene glycol. The immunostimulatory agent may comprise bacterial cell components, nucleic acids, and cytokines. In particular, bacterial cell wall components, LPS, bacterial DNA, viral RNA, CpG oligonucleotides, double-stranded RNA, β-glucan, zymosan, IL-2, IL-6, IL-7, IL-15, IFN-γ, IFN-α and GM-CSF are contemplated. The plurality of tumor cell antigens may comprise a tumor cell lysate, for example, from a breast cancer cell, a head & neck cancer cell, a lung cancer cell, a stomach cancer cell, an esophageal cancer cell, a skin cancer cell, a colon cancer cell, an ovarian cancer cell, a prostate cancer cell, a testicular cancer cell, a uterine cancer cell, a cervical cancer cell, a pancreatic cancer cell, or a liver cancer cell. The composition may administered to said subject once or more than once, for example, the composition may be administered to said subject 2, 3, 4, 5, 6, 7, 8, 9 or 10 times. The subject may suffer from recurrent cancer, metastatic cancer, or multi-drug resistant cancer. The method may further comprise administering to said subject a second cancer therapy. The second cancer therapy may be gene therapy, other immunotherapy, brachytherapy, chemotherapy, radiotherapy, toxin therapy, or hormonal therapy.

In another embodiment, there is provided a composition of matter comprising (a) a biocompatible polymer; (b) a plurality of tumor cell antigens; and (c) an immunostimulatory agent. The composition may further comprise a pharmaceutically acceptable buffer, diluent or excipient. The biocompatible polymer may comprise silk, elastin, chitin, chitosan, poly(d-hydroxy acid), poly(anhydrides), and poly(athoesters). More particularly, the biocompatible polymer may comprises polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy)methane] (PCPM), copolymers of SA, CPP and CPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, polylactide-co-glycolide, polylactic acid, and polyethylene glycol. The immunostimulatory agent may comprise bacterial cell components, nucleic acids, and cytokines. In particular, bacterial cell wall components, LPS, bacterial DNA, viral RNA, CpG oligonucleotides, double-stranded RNA, β-glucan, zymosan, IL-2, IL-6, IL-7, IL-15, IFN-γ, IFN-α and GM-CSF are contemplated. The plurality of tumor cell antigens may comprise a tumor cell lysate, for example, derived from a breast cancer cell, a head & neck cancer cell, a lung cancer cell, a stomach cancer cell, an esophageal cancer cell, a skin cancer cell, a colon cancer cell, an ovarian cancer cell, a prostate cancer cell, a testicular cancer cell, a uterine cancer cell, a cervical cancer cell, a pancreatic cancer cell, or a liver cancer cell. The polymer may be polylactide-co-glycolide and the immunostimulatory agent is CpG oligonucleotide. The polymer may be polylactic acid and polyethylene glycol, and the immunostimulatory agent is CpG. These compositions may further comprise GM-CSF.

In yet another embodiment, there is provided a kit comprising (a) a biocompatible polymer; (b) a plurality of tumor cell antigens; and (c) an immunostimulatory agent, each of (a)-(c) being disposed in a discrete container. The kit may further comprise a pharmaceutically acceptable buffer, diluent or excipient. The biocompatible polymer may comprise silk, elastin, chitin, chitosan, poly(d-hydroxy acid), poly(anhydrides), and poly(athoesters). More particularly, the biocompatible polymer may comprises polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis(p-carboxypheonoxy) methane] (PCPM), copolymers of SA, CPP and CPM, poly(amino acids), poly(pseudo amino acids), polyphosphazenes, derivatives of poly[(dichloro)phosphazenes] and poly[(organo) phosphazenes], poly-hydroxybutyric acid, or S-caproic acid.polylactide-co-glycolide, polylactic acid, and polyethylene glycol. The immunostimulatory agent may comprise bacterial cell components, nucleic acids, and cytokines. In particular, bacterial cell wall components, LPS, bacterial DNA, viral RNA, CpG oligonucleotides, double-stranded RNA, β-glucan, zymosan, IL-2, IL-6, IL-7, IL-15, IFN-γ, IFN-α and GM-CSF are contemplated. The plurality of tumor cell antigens may comprise a tumor cell lysate, for example, derived from a breast cancer cell, a head & neck cancer cell, a lung cancer cell, a stomach cancer cell, an esophageal cancer cell, a skin cancer cell, a colon cancer cell, an ovarian cancer cell, a prostate cancer cell, a testicular cancer cell, a uterine cancer cell, a cervical cancer cell, a pancreatic cancer cell, or a liver cancer cell.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1—Tumor growth (mm³) plotted against time in days. Mice were inoculated with 5×10⁵ syngeneic melanoma cells and four days later vaccinated in the following groups: Control—No vaccine, GM+CpG+XR−B16—GM-CSF secreting bystander cells plus 100 μg CpG 1826 plus irradiated B 16 tumor cells, [PLGA+CpG+GM]+XR−B16—microparticles loaded with CpG and GM-CSF admixed with irradiated tumor cells and [PLGA+CpG+GM+TL]-microparticles loaded with CpG, GM-CSF and tumor lysate. The group of mice receiving the microparticles loaded with tumor lysate and immune-stimulatory agents displayed the slowest tumor growth and longest survival.

FIG. 2—T cell proliferation assay. Nine days following vaccination with one of the vaccine groups described above, splenocytes were harvested and cultured for 7 days in vitro. CFSE staining was performed and CD8+ T-cells undergoing proliferation were detected by flow cytometry as identified by dilution of CFSE. As illustrated, mice that received microparticles containing both CpG and tumor lysate underwent vigorous T cell proliferation with 72.7% of the T-cells having proliferated in response to the vaccine.

### DETAILED DESCRIPTION OF THE PREFERRED EMBODIMENTS

I. The Present Invention

As discussed above, there is a need for improved therapies of multiple diseases including cancer. Many tumors are ineffectively treated by standard treatment strategies including surgery, chemotherapy and radiation therapy. Immunotherapy, and specifically tumor vaccination, constitute an as of yet unrealized approach that has great potential due to its specificity and lack of toxicity. A primary concern is the inability to deliver the proper signals and antigens for vaccination when attempting to shape the appropriate immune response. Fabricated encapsulated microparticles offer a means of overcoming these limitations as they enable preferential uptake by antigen presenting cells, are non-toxic to cells, protect the packaged material from destruction, and provide sustained release of antigen, negating the requirement for repeated dosings or boosters. In addition they can be packaged, scaled up, and easily stored.

The inventors have developed microparticles for tumor vaccine therapy by loading them with tumor cell lysate and immunostimulatory agents for induction of potent, effective immunity against the targeted tumor in both prophylactic and therapeutic tumor models. One benefit of this approach is the ability to load multiple antigens from a single autologous tumor or multiple tumors in the context of the ideal immunostimulatory agents or agents to the antigen presenting cells of interest. One of the major problems with inducing adequate immunity against tumors is the lack of adequate tumor antigens and the inefficient presentation of antigens to antigen presenting cells. This present invention overcomes these shortcomings as tumor lysate contains multiple tumor antigen epitopes and the dendritic cells and macrophages themselves will phagocytose the microparticles that are loaded with the appropriate immunomodulating agents. Moreover, there is a continual release of antigen from the microparticles, thereby sustaining and furthering the immune response. In pilot studies, the inventors have found these microparticles to be more effective than attenuated whole tumor cell or peptide vaccination in their ability to suppress established tumor growth and induce tumor-specific cellular immunity. They also should be superior to the delivery of antigens using coated devices with surface-bonded antigens, which do not provide sustaained release of antigen. This strategy could be used for vaccination against multiple tumor types and possibly against infectious diseases as well.

II. Tumor Cell Lysates

In accordance with the present invention, there is provided a tissue lysate derived from cancer cells, cancerous tissue or tumor. One source of cancer cells/tumor lysates is the patient to be treated or even from a bank of similar tumors form multiple patients. Generally, standard biopsy procedures can be used to obtain samples from solid tumors that can then be lysed to produce tumor lysates. Biopsy procedures will generally involve the sterility required of surgical operations, even though the tissues being sample are from cadavers or animals that will be sacrificed. For internal tissues, biopsies can be performed percutaneously with or withour radilogic guidance or via incisions that will be made proximal to the tissue of interest, followed by retraction, excision of tissue and surgical closing of the incision. Superficial tissue sites are accessed by simple excision of the available tissue. Appropriate physiologic buffers are generally applied to the tissue, or the tissues are immersed therein. The tissue may also be cooled to appropriate temperatures for limited periods of time. Steps should be taken to ensure that apoptosis or other cellular degredation will not be induced in the tissue specimen. Cancer cells such as leukemias can be dealt with by purification of cells from blood using affinity procedures.

Physical methods may also be employed to disrupts the cells, such as freeze-thawing, sonication, shearing, irradiation or exposure to microwaves.

A variety of detergents may be used to solubilize cells, including anionic, cationic, zwitterionic and non-ionic detergents. By virtue of their amphipathic nature, detergents are able to disrupt bipolar membranes. In selecting a detergent, consideration will be given to the nature of the target antigen(s), and the fact that anionic and cationic detergents are likely to have a greater effect on protein structure than zwitterionic or non-ionic detergents. However, non-ionic detergents tend to interfere with charge-bases analyses like mass spectroscopy, and are also suspectible to pH and ionic strength. Zwitterionic detergents provide intermediate properties that, in some respects, are superior to the other three detergent types. Offering the low-denaturing and net-zero charge characteristics of non-ionic detergents, zwitterionics also efficiently disrupt protein aggregation without the accompanying drawbacks. Exemplary anionic detergents include chenodeoxycholic acid, N-lauroylsarconsine sodium salt, lithium dodecyl sulfate, 1-octanesulfonic acid sodium salt, sodium cholate hydrate, sodium deoxycholate, sodium dodecyl sulfate and glycodeoxycholic acid sodium salt. Cationic detergents include cetylpyridinium chloride monohydrate and hexadecyltrimethylammonium bromide. Zwitterionic detergents include CHAPS, CHAPSO, SB3-10 and SB3-12. Non-ionic detergents may be selected from N-decanoyl-N-methylglucamine, digitonin, n-dodecyl β-D-maltoside, octyl α-D-glucopyranoside, Triton X-100, Triton X-114, Tween 20 and Tween 80.

Commercial sources of tumor lysates also are available. For example, Protein Biotechnologies (www.proteinbiotechnologies.com/) sells lung, breast, colon, uterine, cervical, ovarian and stomach tumor lysates.

III. Immunomodulating Agents

A variety of agents may be incorporated into the vaccine compositions of the present invention to act as immunostimulatory agents. These generally fall into the categories of bacterial cell products, nucleic acids, cytokines and growth factors, and miscellaneous agents.

A. Bacterial Cell Products

Bacterial cell wall components such as lipopolysaccharide (LPS), also termed endotoxin, peptidoglycan (PG), lipoteichoic acid (LTA), and lipopeptides/proteins (LP) represent such bacterial compounds which are present in Gram-negative and/or Gram-positive bacteria. They are able to activate cells of the innate and adaptive immune system, but also react with further cells like vascular cells and epithelial cells. Bacterial DNA also is able to stimulate immune response, as discussed below.

B. Nucleic Acids

Unmethylated CpG motifs are prevalent in bacterial but are rare in vertebrate genomes. Oligodeoxynucleotides containing CpG motifs activate host defense mechanisms leading to innate and acquired immune responses. The recognition of CpG motifs requires Toll-like receptor (TLR) 9. Cells that express TLR-9, which include plasmacytoid dendritic cells (PDCs) and B cells, produce proinflammatory cytokines, interferons, and chemokines. CpG-driven innate immunity protects against challenge with a wide variety of antigens, including pathogens, allergens and cancer cells. Thus, CpG ODNs enhance the development of acquired immune responses in vaccination. See also U.S. Pat. Nos. 6,821,957, 6,653,292, 6,429,199, 6,406,705, 6,339,068, 6,239,116, 6,214,806, 6,207,646 and 6,194,388.

Other nucleic acids that have immunostimulatory properties include bacterial DNA, viral RNA, and double-stranded RNA. Bacterial DNA is immunostimulatory largely due to unmethylated CpG motifs.

C. Cytokines

A variety of cytokines, interferons and other factors can be used to enhance the immune response to tumor antigens of the present invention. For example, granulocyte-macrophage colony-stimulating factor (GM-CSF) is a colony-stimulating factor that stimulates the production of white blood cells, especially granulocytes and macrophages, and cells (in the bone marrow) that are precursors of platelets. It is a cytokine that belongs to the family of drugs called hematopoietic (blood-forming) agents. It is also referred to as sargramostim.

Other cytokines that may be used in accordance with the present invention are IL-2, IL-6, IL-7, IL-15, IFN-γ, IFN-α, although this is not a limiting list.

D. Miscellanous Agents

A variety of other immunomodulatory agents also may be used in accordance with the present invention. β-glucans are polysaccharides generally come from cultured extract of Baker's yeast cell wall. They are found bound together as a sugar/protein complex. Certain plants and microorganisms are naturally high in these polysaccharides. The richest concentrated source is Baker's yeast cell walls, but it also is present in lesser amounts in mushroom extracts and lentinen, barley, oat, etc. Sodium alginate is also an excellent source, but the high sodium content is a major drawback in the processing for supplemental use.

Another form of β-glucan is a research extract called Zymosan™ (Biosynth) Zymosan. It is mannan-rich and prepared according to Pillemer et al. (1956). Zymosan™ activates the alternative complement cascade. It becomes coated with C3b/C3bi and is therefore a convenient opsonized particle. It also leads to C5a—production in serum. It is a potent stimulator of alveolar macrophages. It induces the release of cytokines, e.g., interleukin 8 (IL-8) from human neutrophils and proinflammatory cytokines in immune cells. The toll-like receptor 2 has been shown to be involved in Zymosan™ induced signaling. Zymosan™ also induces protein phosphorylation and inositol phosphate formation.

IV. Microparticle Delivery Systems

In accordance with the present invention, polymer-based microparticles are used to delivery tumor antigens and immunomodulatory agents of the present invention. A variety of polymer based microparticles can be employed in this context.

Polylactide-co glycolide (PLGA) biodegradable polymers serve as the structural matrix in which medication is incorporated in the long-term delivery systems. The final products of PLGA degradation are lactic acid and glycolic acid, which are water soluble, non-toxic products of normal metabolism. See also U.S. Pat. Nos. 6,884,435, 5,603,960 and 6,913,767.

Polylactic acid (poly-lactide; PLA) is a polymer known for its ability to biodegrade. Since it does biodegrade, and can be processed to have such a wide variety of properties, it can be used in everything from packaging to surgical sutures. It also finds uses as resorbable microspheres and implants for the delivery of drugs and vaccines.

Polyethylene glycol (PEG) is a water-soluble, waxy solid that is used extensively in the cosmetic and toiletry industry. As the molecular weight of PEG increases, viscosity and freezing point increase. Although PEG is water soluble, solubility is greatly reduced at temperatures approaching 0° C., allowing experiments to run for 15-20 minutes before dissolution of PEG becomes pronounced. At higher temperatures (above 10° C.) this length of time is much shorter.

The following U.S. patent describe various polymers for use in microparticle applications according to the present invention: U.S. Pat. Nos. 6,884,435, 6,565,777, 6,534,092, 6,528,087, 6,379,704, 6,309,569, 6,264,987, 6,210,707, 6,090,925, 6,022,564, 5,981,719, 5,871,747, 5,723,269, and 5,578,709.

V. Preparing Polymer-Antigen Complexes

Microparticles were prepared from PLGA using an oil-in-water solvent evaporation method. A commonly used emulsion stabilizer in the solvent evaporation method for PLGA microparticle preparation is partially hydrolyzed PVA, which is a copolymer of poly (vinyl acetate) and poly (vinyl alcohol). The inventors chose an 88% hydrolysed PVA because a study by Murakami et al. (1997) found this to be the optimum degree of hydrolyzation of PVA for the manufacture of nano/microparticles. The irreversible binding of PVA on the microparticle surface is likely to happen when the organic solvent is removed from the interface in which interpenetration of PVA and PLGA molecules occur. The inventors have demonstrated the ability to control the size of particles prepared from PLGA, where particle size is governed by the stirring rate and the PVA concentrations of the continuous phase. This is important because it has been found that there is a direct relationship between the degradation rate and particle size. In smaller particles, degradation products formed within the particle can diffuse easily to the surface, while in larger particles degradation products have a longer path to the surface of the particle, during which autocatalytic degradation of the remaining polymer can occur.

More specifically, the oil-in-water solvent evaporation technique involves the use of three phases: (1) an inner water phase containing the immunostimulatory molecules and tumor lysates to be incorporated; (2) an intermediate organic phase consisting of a polymer/methylene chloride solution; and (3) an outer water phase containing an emulsifying agent. Particles are collected by centrifugation. The particles are then resuspended in 10 mM Tris-HCl, 1 mM EDTA, pH 7.5 (TE) buffer.

Other methods for the preparation of microparticles include solvent extration/evaporation techniques, double coacervation, super-critical CO₂, electrohydrodynamic preparation, spray drying, jet spraying and micromixer preparation.

VI. Methods of Therapy

A. Treating Cancer

The present invention also involves the treatment of cancer. The types of cancer that may be treated not limited other than that they be responsive to immunotherapy according to the present invention. Thus, it is contemplated that a wide variety of tumors may be treated using the immunotherapy of the present invention, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra. Of particular interest is direct intratumoral administration, perfusion of a tumor, or admininstration local or regional to a tumor, for example, in the local or regional vasculature or lymphatic system, or in a resected tumor bed.

The immunotherapeutic compsition may also be administered parenterally or intraperitoneally. Solutions can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

B. Combined Therapy with Chemotherapy or Radiotherapy

Tumor cell resistance to traditional therapies represents a major problem in clinical oncology. One goal of current cancer research is to find ways to improve the efficacy of chemo- and radiotherapy. One way is by combining such traditional therapies with a new therapy. For example, the herpes simplex-thymidine kinase (HS-tk) gene, when delivered to brain tumors by a retroviral vector system, successfully induced susceptibility to the antiviral agent ganciclovir (Culver et al., 1992). In the context of the present invention, it is contemplated that the immunotherapy could be used similarly in conjunction with chemo- or radiotherapeutic intervention. It also may prove effective to combine immunotherapy of the present invention with chemotherapy and/or radiotherapy, as described below.

To kill cells, inhibit cell growth, inhibit metastasis, inhibit angiogenesis or otherwise reverse or reduce the malignant phenotype of tumor cells, using the methods and compositions of the present invention, one would generally administer the immunotherapeutic composition of the present invention and at least one other agent. These compositions would be provided in a combined amount effective to kill or inhibit proliferation of the cell. This process may involve administering the immunotherapeutic composition and the other agent(s) or factor(s) at the same time. This may be achieved by administering a single composition or pharmacological formulation that includes both agents, or by administering two distinct compositions or formulations, at the same time, wherein one composition includes the immunotherapeutic compositions and the other includes the other agent.

Alternatively, the immunotherapy treatment may precede or follow the other agent treatment by intervals ranging from minutes to weeks. In embodiments where the other agent and immunotherapuetic composition are applied separately to the cell, one would generally ensure that a significant period of time did not expire between the time of each delivery, such that the agent and the immunotherapy composition would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the immunotherapeutic composition or the other agent will be desired. Various combinations may be employed, where the immunotherapeutic composition is “A” and the other agent is “B,” as exemplified below:

| | | | | | | | |
|---|---|---|---|---|---|---|---|
| A/B/A | B/A/B | B/B/A | A/A/B | B/A/A | A/B/B | B/B/B/A | B/B/A/B |

| | | | | | | |
|---|---|---|---|---|---|---|
| A/A/B/B | A/B/A/B | A/B/B/A | B/B/A/A | B/A/B/A | B/A/A/B | B/B/B/A |
| A/A/A/B | B/A/A/A | A/B/A/A | A/A/B/A | A/B/B/B | B/A/B/B | B/B/A/B |

Other combinations are contemplated. Again, to achieve cell killing, both agents are delivered to a cell in a combined amount effective to kill the cell.

Agents or factors suitable for use in a combined therapy are any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide.

In treating cancer according to the invention, one would contact the tumor cells with an agent in addition to the immunotherapeutic compsition. This may be achieved by irradiating the localized tumor site with radiation such as X-rays, UV-light, γ-rays or even microwaves. Alternatively, the tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or more preferably, cisplatin. The agent may be prepared and used as a combined therapeutic composition, or kit, by combining it with the immunotherapeutic compsition, as described above.

Agents that directly cross-link nucleic acids, specifically DNA, are envisaged to facilitate DNA damage leading to a synergistic, antineoplastic combination with the immunotherapeutic compsition. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the local or regional delivery of the immunotherapeutic compsition to patients with cancer will be a very efficient method for treating the clinical disease. Similarly, the chemo- or radiotherapy may be directed to a particular, affected region of the subjects body. Alternatively, systemic delivery of the immunotherapeutic compsition and/or the agent may be appropriate in certain circumstances, for example, where extensive metastasis has occurred.

In addition to combining immunotherapies with chemo- and radiotherapies, it also is contemplated that combination with gene therapies will be advantageous. For example, any tumor-related gene conceivably can be targeted in combination with the immunotherapy, for example, p21, Rb, APC, DCC, NF-1, NF-2, BCRA2, p16, FHIT, WT-1, MEN-I, MEN-II, BRCA1, VHL, FCC, MCC, ras, myc, neu, raf, erb, src, fms, jun, trk, ret, gsp, hst, bcl and abl.

C. Formulations

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render compositions stable and allow for administration. Aqueous compositions of the present invention comprise an effective amount of the immunotherapeutic compsition to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the immunotherapeutic compsition of the present invention, its use in therapeutic compositions is contemplated. Supplementary active ingredients also can be incorporated into the compositions.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

VII. Kits

Generally, kits comprises separate vials or containers for the various reagents, such as polymers, tumor lysates, immunostimulatory agents, antibodies, etc. The reagents are also generally prepared in a form suitable for preservation by dissolving in a suitable solvent, e.g., lyophilized. Examples of suitable solvents include water, ethanol, various buffer solutions, and the like. The various vials or containers are often held in blow-molded or injection-molded plastics.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials & Methods

Murine tumor cell line. B16(F1) (ATCC) is a murine melanoma derived from C57BL/6 mice. Culture media for the B16(F1) is Dulbecco's Modified Eagle's Medium (DMEM) supplemented with 2 mM L-glutamine, 1% penicillin/streptomycin, 1 mM sodium bicarbonate and 10% heat-inactivated FBS. The murine melanoma model was used for evaluation of the vaccine strategy.

Tumor model and vaccine protocol. Wild-type tumor cells (1×10⁵-3×10⁶), were subcutaneously implanted into the hind leg of syngeneic mice (6-8 weeks old, Jackson Labs, Bar Harbor, Me.). Measurement of tumor development and growth was documented every other day with calipers and volumes determined as width²×length×0.52 cm³. Mice were vaccinated intraperitoneally with microparticles as described below 4 days after initial tumor cell challenge and again 7 days later.

Anesthetic agents and animal care. All mice are anesthetized using Halothane inhalation (Halocarbon Labs, N.J.) during inoculation. All animals are housed under standard conditions in accordance with our institution's animal care and use committee, which follows the U.S. Public Health Service's guide for the care and use of animals. Mice were sacrificed if tumor size was greater than 2.5 cm in longest dimension or if mice assume a “sick mouse posture”.

Vaccine preparation. PLGA microparticles were formulated by loading the particles with combinations of tumor cell lysate, CpG oligonucleotides, Alum and GM-CSF. Tumor cell lysates were prepared by freeze thawing B16 tumor cells three times for 5 minutes each and then irradiating the lysate with 20Gy. The CpG oligodeoxynucleotides (ODN) are phosphorothioate-modified. The following sequence was used (CG dinucleotides indicated): CpG ODN 1826: 5′TCCATGACGTTCCTGACGTT′3 (SEQ ID NO:1) No endotoxin is detected in ODN preparations (<0.03 EU/ml; LAL-assay; BioWhittaker, Walkersville, Md.). CpG ODN were purchased from Coley Pharmaceutical group. Recombinant mouse GM-CSF was purchased from R&D systems and similarly loaded into the microparticles. Alum was obtained from Sigma Chemical and used as a control immune stimulant.

Cell assays of activation. Nine days following vaccination of naive mice, splenocytes were harvested for cellular studies to determine T-cell proliferation and IFN-g secretion and compared to unvaccinated controls. Various formulations of the loaded microparticles were compared to controls.

T-cell proliferation assay. Splenocytes at 1×10⁷/ml in PBS are incubated with CFSE (5(6)-Carboxy fluorescein diacetate N-succinimidyl ester) at a concentration of 2 μM at room temperature for 10 minutes. Staining is terminated by adding culture medium containing 10% heat inactivated fetal calf serum. The cells are washed three times with PBS containing 1% fetal calf serum and re-suspended in culture medium at 2×10⁶/ml. Stained cells are cultured in 12-well tissue culture plate with or without precoated irradiated tumor cells. After 7 days incubation, cells are harvested and stained with anti-CD4 and anti-CD8 mAb, and then subjected to flow cytometry analysis.

Intracellular Staining and Flow Cytometry Analysis. Splenocytes were incubated with unlabeled primary rabbit mAb of interest for 1 h at 4° C. (Receptors of interest include: T-cell: CD4, CD8 and intra-cellular staining for IFN-γ. Cells are analyzed immediately following staining on a FACScan (Becton Dickinson, San Jose, Calif.). Fresh or cultured cells are labeled with fluorochrome-conjugated mAbs specific for different surface markers for 20 minutes at 4° C. After binding and washing, cells are fixed with formalin in PBS for flow cytometry analysis. For intracellular staining, cells are first stained with surface markers, and then fixed and permeabilized with fixation and permeabilization buffers respectively. Cells are then stained with PE-conjugated IFN-γ for 20 minutes at room temperature in the dark. After washing away excessive antibodies with permeabilization buffer, cells are resuspended in staining buffer for flow cytometry analysis. Data as analyzed using CellQuest^{Pro} software. Quadrant markers are set according to isotype control antibodies.

### Example 2

### Results

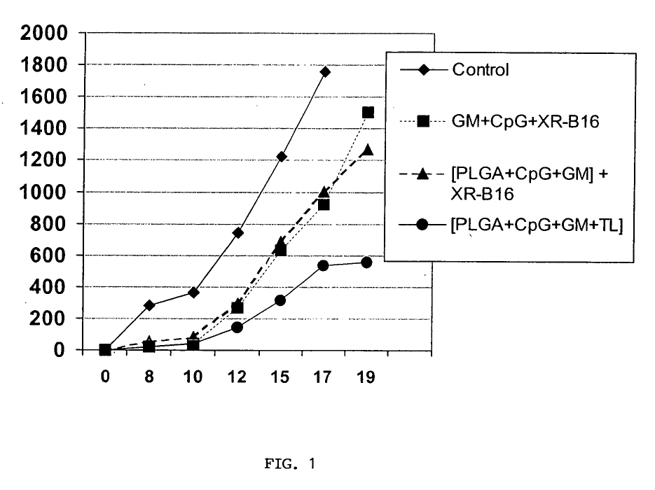

Mice were inoculated with syngeneic melanoma cells and vaccinated four days later. The group of mice receiving the microparticles loaded with tumor lysate and immune-stimulatory agents displayed the slowest tumor growth and longest survival (FIG. 1).

Mice were vaccinated with microparticles containing various combinations of polyers, immunostimulatory agents, and lysates. As shown in Table 1, mice receiving the microparticles loaded with CpG and tumor lysate with or without GM-CSF had the greatest number of CD8+ IFN-γ secreting T-cells.

TABLE 1

| GROUP | % CD8+ IFN-γ+ cells of T-cells |
|---|---|
| PLGA | 10.9 |
| PLGA + CpG | 7 |
| PLGA + Alum + TL | 6.1 |
| PLGA + GM-CSF + TL | 11.8 |
| PLGA + CpG + TL | 20.7 |
| PLGA + GM-CSF + CpG + TL | 25.3 |

Mice were vaccinated with one of the following groups:
PLGA - microparticles only;
PLGA + CpG - microparticles loaded with CpG;
PLGA + Alum + TL - microparticles loaded with alum and tumor lysate;
PLGA + GM-CSF + TL - microparticles loaded with GM-CSF and tumor lysate;
PLGA + CpG + TL - microparticles loaded with CpG and tumor lysate and
PLGA + GM-CSF + CpG + TL - microparticles loaded with GM-CSF, CpG and tumor lysate. Splenocytes were harvested 9 days after vaccination and cultured overnight with irradiated tumor cells.

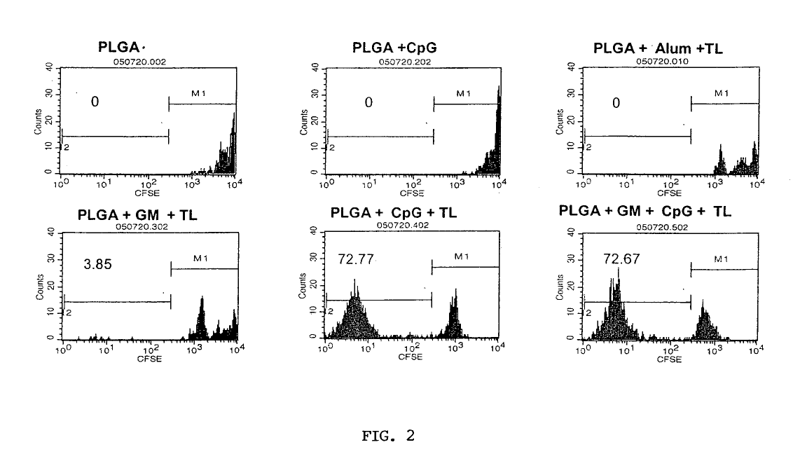

Following vaccination with one of the vaccine groups described above, splenocytes were harvested and cultured for 7 days in vitro. As seen in FIG. 2, mice that received microparticles containing both CpG and tumor lysate underwent vigorous T-cell proliferation, with 72.7% of the T-cells having proliferated in response to the vaccine.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

IX. References

The following references, to the extent that they provide exemplary procedural or other details supplementary to those set forth herein, are specifically incorporated herein by reference:
U.S. Pat. No. 5,578,709
U.S. Pat. No. 5,603,960
U.S. Pat. No. 5,723,269
U.S. Pat. No. 5,871,747
U.S. Pat. No. 5,981,719
U.S. Pat. No. 6,022,564
U.S. Pat. No. 6,090,925
U.S. Pat. No. 6,194,388
U.S. Pat. No. 6,207,646
U.S. Pat. No. 6,210,707
U.S. Pat. No. 6,214,806
U.S. Pat. No. 6,239,116
U.S. Pat. No. 6,264,987
U.S. Pat. No. 6,309,569
U.S. Pat. No. 6,339,068
U.S. Pat. No. 6,379,704
U.S. Pat. No. 6,406,705
U.S. Pat. No. 6,429,199
U.S. Pat. No. 6,528,087
U.S. Pat. No. 6,534,092
U.S. Pat. No. 6,565,777
U.S. Pat. No. 6,653,292
U.S. Pat. No. 6,821,957
U.S. Pat. No. 6,884,435
U.S. Pat. No. 6,884,435
U.S. Pat. No. 6,913,767
Culver et al., Science, 256(5063):1550-1552, 1992.
Murakami et al., Am. J. Physiol., 272:L197-L202, 1997.
Pillemer et al., J. Exp. Med., 103(1):1-13, 1956.
Remington's Pharmaceutical Sciences, 15^{th} ed., 33:624-652, Mack Publishing Company, Easton, Pa., 1980.
Remington's Pharmaceutical Sciences” 15^{th} Edition, pages 1035-1038 and 1570-1580, 1990.

What is claimed is:

1. A method of treating or preventing cancer in a subject comprising administering to said subject a composition comprising a biocompatible polymer, a plurality of tumor cell antigens and an immunostimulatory agent, wherein said plurality of tumor antigens and said immunostimulatory agent are encapsulated in said polymer.

2. The method of claim 1, wherein said method comprises treating cancer.

3. The method of claim 1, wherein said method comprises preventing cancer.

4. The method of claim 1, wherein said biocompatible polymer is biodegradable.

5. The method of claim 1, wherein said biocompatible polymer is silk, elastin, chitin, chitosan, poly(d-hydroxy acid), poly(anhydrides), and poly(athoesters).

6. The method of claim 1, wherein said biocompatible polymer comprises polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid.

7. The method of claim 1, wherein said immunostimulatory agent comprises bacterial cell wall, LPS, bacterial DNA, viral RNA, CpG oligonucleotides, double-stranded RNA, β-glucan, zymosan, GM-CSF IL-2, IL-6, IL-7, IL-15, IFN-γ, IFN-α.

8. The method of claim 1, wherein said plurality of tumor cell antigens comprises a tumor cell lysate.

9. The method of claim 8, wherein the tumor cell lysate is derived from a breast cancer cell, a head & neck cancer cell, a lung cancer cell, a stomach cancer cell, an esophageal cancer cell, a skin cancer cell, a colon cancer cell, an ovarian cancer cell, a prostate cancer cell, a testicular cancer cell, a uterine cancer cell, a cervical cancer cell, a pancreatic cancer cell, or a liver cancer cell.

10. The method of claim 8, wherein the tumor lysate is derived from a neuroblastoma, a Wilm's tumor, a rhabdoid tumors, a sarcomas (osteogenic or non-osteogenic), a hepatoblastomas, a rhabdomyosarcomas, a lymphomas, or a leukemia.

11. A composition of matter comprising a (a) biocompatible polymer and (b) a plurality of tumor cell antigens and an immunostimulatory agent encapsulated in said biocompatible polymer.

12. The composition of claim 11, wherein said biocompatible polymer is biodegradable.

13. The composition of claim 11, wherein said immunostimulatory agent comprises bacterial cell wall, LPS, bacterial DNA, viral RNA, CpG oligonucleotides, double-stranded RNA, β-glucan, zymosan, GM-CSF IL-2, IL-6, IL-7, IL-15, IFN-γ, IFN-α.

14. The composition of claim 11, wherein said plurality of tumor cell antigens comprises a tumor cell lysate.

15. The composition of claim 15, wherein the tumor cell lysate is derived from a breast cancer cell, a head & neck cancer cell, a lung cancer cell, a stomach cancer cell, an esophageal cancer cell, a skin cancer cell, a colon cancer cell, an ovarian cancer cell, a prostate cancer cell, a testicular cancer cell, a uterine cancer cell, a cervical cancer cell, a pancreatic cancer cell, or a liver cancer cell.

16. The composition of claim 11, wherein the polymer is polylactide-co-glycolide and the immunostimulatory agent is CpG oligonucleotide.

17. The composition of claim 11, wherein the polymer is polylactic acid and polyethylene glycol, and the immunostimulatory agent is CpG.

18. The composition of claim 16, further comprising GM-CSF.

19. The composition of claim 17, further comprising GM-CSF.

20. A kit comprising composition comprising a (a) biocompatible polymer and (b) a plurality of tumor cell antigens and an immunostimulatory agent encapsulated in said biocompatible polymer, the composition being disposed in a discrete container.


SEQUENCE LISTING (1 sequence)

SEQ ID NO: 1 - DNA, 20 bp, Artificial Sequence
  Description of Artificial Sequence  Synthetic Primer
tccatgacgttcctgacgtt